(19) United States
(12) Patent Application Publication
(10) Pub. No.: US 2010/0191070 A1
(43) Pub. Date: Jul. 29, 2010

(54) DIAGNOSTIC METHOD INCLUDING UNIFORM ENCODING OF INFORMATION

(76) Inventor: Keat Jin Lee, Guilford, CT (US)

Correspondence Address: MICHAUD-Kinney Group LLP, 306 INDUSTRIAL PARK ROAD, SUITE 206, MIDDLETOWN, CT, 06457, US

(21) Appl. No.: 12/694,578

(22) Filed: Jan. 27, 2010

Related U.S. Application Data
(63) Continuation of application No. 11/799,269, filed on Apr. 30, 2007.
(60) Provisional application No. 60/796,678, filed on May 1, 2006.

(51) Int. Cl. A61B 5/00

(52) U.S. Cl. 600/300

(57) ABSTRACT

A diagnostic encoding method is provided. The method includes assigning a first plurality of identifiers to an anatomical structure of the human body and assigning a second plurality of identifiers to diagnostic criterion by which caregivers evaluate patients. The method further includes, when diagnosing a patient, recording findings of the diagnosis of the patient using at least one of each of the first plurality and the second plurality of identifiers. In one embodiment, the first plurality of identifiers includes a first subset of identifiers identifying each organ in the human body (e.g., a right ear) and a second subset of identifiers identifying component parts of each of the organs in the first subset (e.g., an ear drum of the right ear). In one embodiment, the second plurality of identifiers includes a third subset of identifiers identifying symptoms relating to the first and the second identifiers.

### CROSS-REFERENCE TO RELATED APPLICATION

This application is a continuation application of U.S. patent application Ser. No. 11/799,269 filed Apr. 30, 2007, incorporated herein by reference in its entirety.

### BACKGROUND OF THE INVENTION

1. Field of the Invention

This invention relates generally to a medical diagnostic method and, more particularly, to diagnostic systems and methods employing a dynamic and uniform method of encoding a patient's symptoms as they pertain to each organ within the human body.

2. Description of the Related Art

It is well known in the medical community to use alphanumeric or other codes to describe a patient's medical condition and/or course of treatment. For example, the International Classification of Diseases, 9th Revision, Clinical Modification (ICD9-CM) is published annually by the U.S. government and serves as a reference within the health care and related professions. The ICD9-CM provides numeric codes for describing a “clinical history” of a patient. Generally speaking, the ICD9-CM codes provide a standard for communication medical condition and treatment such as, for example, to facilitate reimbursement by insurance companies of health services, evaluation of utilization patterns and epidemiologic research.

Within ICD9-CM, volumes include a Classification of Diseases and Injuries—Tabular Index and—an Alphabetic Index, respectively, and a Classification of Procedures—Tabular Index and—an Alphabetic Index, respectively. The Classification of Diseases and Injuries are used by, for example, physicians to report patients' conditions. The Classification of Procedures is used by, for example, medical records personnel within a hospital and/or physician's office to describe procedures performed on patients. The Classification of Diseases and Injuries is a resource for diagnostic coding in the U.S. It provides a comprehensive classification scheme by organizing diseases into various categories and subcategories and assigning unique numeric or alphanumeric codes. The Classification of Diseases and Injuries also provides, throughout the classification scheme, instructions for matching diseases and codes within the clinical setting and includes medical terms (e.g., symptoms, diseases, conditions, etc.) and corresponding numeric codes.

However, the inventor has recognized that conventional classification schemes such as the ICD9-CM scheme, may be useful for reporting a patient's s clinical history to insurance companies and the like, such schemes are overly burdensome and complicated when used as a method for communication between caregivers. Therefore, the inventor has discovered a need for a dynamic and uniform classification method for a caregiver to record the history of a patient's symptoms and/or the findings of a physical examination for each organ of the human body. Accordingly, the inventive classification method provides for efficient and effective communication of diagnostic findings and treatment between caregivers.

### SUMMARY OF THE INVENTION

The present invention is directed to a method for diagnosing patients. The method includes assigning a first plurality of identifiers to an anatomical structure of the human body, and assigning a second plurality of identifiers to diagnostic criterion by which caregivers evaluate patients. The method further includes, when diagnosing a patient, recording findings of the diagnosis of the patient using at least one of each of the first plurality and the second plurality of identifiers. In one embodiment, the first plurality of identifiers includes a first subset of identifiers identifying each organ in the human body (e.g., a right ear) and a second subset of identifiers identifying component parts of each of the organs in the first subset (e.g., an ear drum of the right ear).

In one embodiment, where the first subset of identifiers is comprised of numeric characters, numeric characters representing organs on a first or right side of the body are comprised of even numeric characters and numeric characters representing organs on a second or left side of the body are comprised of odd numeric characters.

In accordance with one aspect of the invention, the second plurality of identifiers includes a third subset of identifiers identifying symptoms relating to at least one of the first subset of identifies (e.g., organs) and the second subset of identifiers (e.g., component parts of the organs). In one embodiment, the second plurality of identifiers includes a fourth subset of identifiers identifying a first characteristic of the symptoms of the third subset. The fourth subset of identifiers, for example, describes a duration of the symptom as reported by the patient to the caregiver. In one embodiment, the second plurality of identifiers includes a fifth subset of identifiers identifying a second characteristic of the symptoms. The fifth subset of identifiers describes, for example, a characteristic of the start of the symptom (immediate onset) as reported by the patient to the caregiver.

In accordance with another aspect of the invention, the second plurality of identifiers includes a sixth subset of identifiers identifying an observed diagnostic finding (e.g., a perforation) relating to at least one of the first subset and second subset of identifiers. In one embodiment, the second plurality of identifiers includes a seventh subset of identifiers identifying a first qualification of the observed diagnostic finding of the sixth subset. The seventh subset describes, for example, a characteristic of a severity of the diagnostic finding observed by the caregiver. In one embodiment, the second plurality of identifiers includes an eighth subset of identifiers identifying a second qualification of the observed diagnostic finding. The eighth subset of identifiers describes, for example, a characteristic of a location of the diagnostic finding observed by the caregiver.

In accordance with the present invention, the first plurality of identifiers and the second plurality of identifiers are comprised of unique alphanumeric characters and symbols. The alphanumeric characters and symbols are uniformly recognized by caregivers as pertaining to elements of the anatomical structure and the diagnostic criterion for diagnosing patients.

### BRIEF DESCRIPTION OF THE DRAWINGS

The features and advantages of the present invention will be better understood when the Detailed Description of the Preferred Embodiments given below is considered in conjunction with the figures provided.

FIG. 1 depicts a portion of the anatomy of the human body and an inventive method of encoding diagnostic information in accordance with one embodiment of the present invention.

FIG. 2 is a graphical illustration of the inventive diagnostic encoding method of FIG. 1.

### DETAILED DESCRIPTION OF THE PREFERRED EMBODIMENTS

In accordance with the present invention, a dynamic and uniform method is provided for caregivers to record a history of a patient's symptoms for each organ and/or subcategory of organs within the human body. The method also provides a step for recording findings of a diagnosis, for example, a physical examination of an organ and/or a subcategory or component part of that organ. The method defines a manner of mapping the various organs and subcategories pertaining to those organs within a corresponding grid structure.

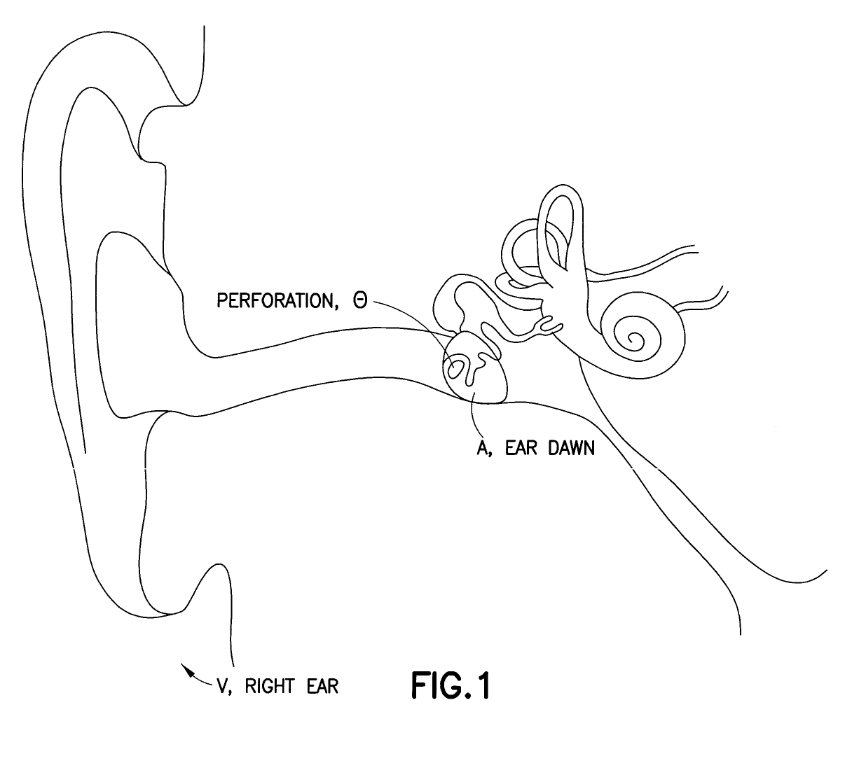

In one embodiment, the grid includes a plurality of identifiers or aliquots such as, for example, seven or more aliquots. In one embodiment, aliquot 1 corresponds to an organ such as the right ear. In accordance with the present invention, each organ is assigned a unique identifier. For example, and as is illustrated in FIG. 1, the right ear is assigned a roman numeral “V,” whereas the left ear (not shown) is assigned a roman numeral “VI.” In one embodiment, organs on the right side of the body are assigned an odd numeral, organs on the left side are assigned even numerals, and organs at a neutral location (e.g., neither on the right or the left side of the body) are assigned an odd numeral. It should be appreciated, however, that the present invention is not limited in this regard as any numeral, symbol or identifier may be employed without departing from the broader aspects of the invention.

Aliquot 2 pertains to a sub-description of an organ such as, for example, a component part of the organ. As noted above, for example, the right ear organ is labeled “V” within aliquot 1. In aliquot 2, an eardrum of the right ear V is assigned, for example, an alphabetic character “A.” Accordingly, if a patient presents with a problem associated with the ear drum of the right ear, in accordance with the present invention, a caregiver employing the inventive encoding method labels the diagnostic area of concern as “V, A.” As should be appreciated from the exemplary analysis, there are as many sub-descriptions or component parts pertaining to a particular organ as are required to allow for a proper designation of the organ and the desired subcategory. For example, the inventive encoding method provides that sub-component and sub-sub-component parts of organs are identifiable using the encoding method. Accordingly, a sub-component part of the ear drum may be identified by, for example, an Aliquot 3.

It should be appreciated that while the inventive encoding method provides for an infinite formulation of identifiers and meanings corresponding to organs of the body, in accordance with the present invention, the designations, subcategories, identifiers and meanings are predefined such that all caregivers employ the same manner of identifying organs, components and sub-components of organs in the human body. In other words, for example, when viewing an encoded diagnostic notation “V, A” all caregivers decode the notation as pertaining to the eardrum of the right ear of the subject patient.

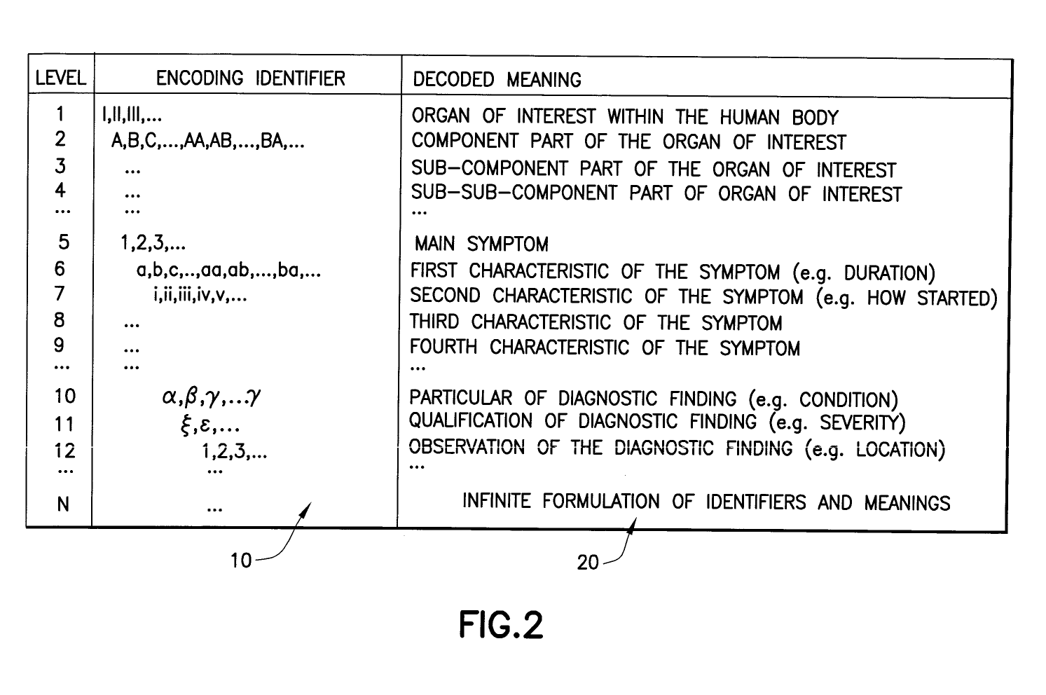

In accordance with one embodiment of the present invention, a graphical illustration of the grid structure of identifiers or aliquots is shown in FIG. 2. The grid structure, shown generally at 10, is depicted in a vertically oriented format as an indented outline, where each indentation further defines the preceding indented item. As illustrated in FIG. 2, aliquot 1 and 2 effectively map or encode the human body in terms of each organ and each sub-description of that organ (e.g., component parts thereof). The decoded meaning of the identifiers is presented generally at 20. As noted above, each organ has a plurality of components, sub-components and sub-sub-components, etc., as is necessary and/or desired to accurately represent the organs of the body. Accordingly, a plurality of aliquot may be employed to accurately represent the organs of the human body. Once the organs and component parts of the organs are defined, the inventive grid structure provides a uniform method for encoding diagnostic findings as follows.

In one embodiment, aliquot 3 describes a main symptom as it relates to the sub-description (aliquot 2) of the organ (aliquot 1), and/or a symptom of the organ (aliquot 1) without reference to the sub-description (aliquot 2). For example, aliquot 3 refers back to aliquot 2 to describe “popping” of the right eardrum. In one embodiment, the “popping” (e.g., barotraumas) symptom is assigned a numeric identifier such as a numeric character “1.” In accordance with the present invention, for example, a caregiver employing the inventive encoding method labels the symptom of a popping in the right eardrum “V, A, 1” as “V” encodes the right ear organ, “A” encodes the ear drum as a sub-description of the ear organ, and “1” encodes the symptom. Conversely, aliquot 3 may relate directly back to aliquot 1 (e.g., the organ directly) so that if a patient complains of a general “popping” in the right ear in this case the caregiver employs the inventive encoding method to label the diagnostic finding “V, 1.”

In one embodiment, aliquot 4 defines a further characteristic of the symptom defined by aliquot 3. For example, in the above-described example aliquot 4 corresponds to a duration of the popping condition. In one embodiment, a plurality of unique identifiers is defined pertaining to durations within a predetermined number of time periods. In the example described, for example, if the patient reports that the popping symptom occurred over a two week period of time, an alphabetic code of “d” is assigned. In this instance, a caregiver employing the inventive encoding method encodes the diagnostic finding “V, A, 1, d”, as “V” encodes the right ear organ, “A” encodes the ear drum as a sub-description of the ear organ, “1” encodes the symptom of popping, and “d” encodes the two week duration of the popping symptom.

In one embodiment, aliquot 5 is employed to further encode one or more details about the symptom. For example, aliquot 5 defines a characteristic describing how the symptom started such as sudden onset, gradual failing, and the like. In the above embodiment, for example, the diagnostic finding “V, A, 1, d” is supplemented by aliquot 5 including a characteristic of the start of the symptom. In this embodiment, a sudden onset is assigned an identified “iii.” Accordingly, the caregiver employing the inventive encoding method encodes the diagnostic finding as “V, A, 1, d, iii” as “V” encodes the right ear organ, “A” encodes the ear drum as a sub-description of the ear organ, “1” encodes the symptom of popping, “d” encodes the two week duration of the popping symptom, and “iii” encodes the sudden onset of the popping condition. It should be appreciated that each symptom has a plurality of characteristics, sub-characteristics, etc., (e.g., third, fourth, fifth, etc.) as is necessary and/or desired to accurately represent the symptom as it pertains to an organ and/or component of the organ. Accordingly, additionally aliquots may define the plurality of symptoms as they pertain to the previous aliquots.

In one embodiment, aliquot 6 describes a diagnostic findings resulting from an examination of the patient in accordance with information gathered and identified in one or more of aliquots 1-5. For example, aliquot 6 describes an observed defect in the organ and/or sub-description such as a perforation or hole in the right eardrum. All such findings are assigned a unique identifier such as, for example, a symbol from the Greek alphabet “θ.” Accordingly, the physical finding of a perforation in the right eardrum need only employ the identifiers from aliquots 1 and 2, as well as aliquot 6, or “V, A, θ” (FIG. 1). It should be appreciated, however, that the inventive diagnostic encoding method also allows for a complete identification of information obtained from the patient. For example, continuing with the above example of a patient presenting with a perforated right eardrum with popping over a two week period and a sudden onset, the inventive diagnostic encoding method records this complete diagnosis “V, A, 1, d, iii, θ”, as “V” encodes the right ear organ, “A” encodes the ear drum as a sub-description of the ear organ, “1” encodes the symptom of popping, “d” encodes the two week duration of the popping symptom, “iii” encodes the sudden onset characteristic of the start of the symptom, and “θ” encodes the perforation as the observed diagnostic finding.

In one embodiment, aliquot 7 is employed to further describe and/or qualify the diagnostic findings in aliquot 6. For example, aliquot 7 encodes an approximation as to the severity of the observed defect. All such qualifications are assigned a unique identifier such as, for example, a symbol from the Greek alphabet “ζ”. In one embodiment, the severity is expressed as a percentage of the organ or sub-description (component part) of the organ that is impaired. For example, if a thirty percent (30%) perforation is observed in the right eardrum of the patient, a caregiver employing the inventive diagnostic encoding method encodes the condition using aliquots 1, 2, 6 and 7 to describe a 30% perforation in the right eardrum as “V, A, θ, ζ”, where “V” encodes the right ear organ, “A” encodes the ear drum as a sub-description of the ear organ, “θ” encodes the perforation as the observed physical finding, and “ζ” encodes the 30% severity.

In one embodiment, aliquot 8 corresponds to a location of the observed physical condition on the organ and/or sub-description (e.g., the perforation in the right eardrum). For example, if a patient presents with a perforation in the posterior superior quadrant, a caregiver employing the inventive diagnostic encoding method assigns the location a numeric character “4.” Continuing with the example outlined above, the caregiver uses the inventive diagnostic encoding method to encode the patient presenting with a right eardrum having a 30% perforation in the posterior superior quadrant as “V, A, θ, ζ, 4”, where “V” encodes the right ear organ, “A” encodes the ear drum as a sub-description of the ear organ, “θ” encodes the perforation as the observed physical finding, “ζ” encodes the 30% severity, and “4” encodes the location of the condition within the posterior superior quadrant.

Referring again to FIGS. 1 and 2, while a first subset of aliquots (e.g., aliquots 1 and 2) effectively map the human body in terms of each organ, sub-description and/or sub-component of that organ, a second subset of aliquots (e.g., aliquot 3-8 and more) map a particular patient's diagnostic findings. For example, and as described above, aliquot 3 describes a main symptom (e.g., popping), aliquot 4 defines a first characteristics of the symptom (e.g., a duration of the popping condition), aliquot 5 describes a second characteristic of the symptom (how the condition started, sudden onset), aliquot 6 describes a physical findings resulting from an examination of the patient (an observed defect, a perforation), aliquot 7 describes a characteristic of the physical findings (e.g., a severity of the observed defect, 30%), and aliquot 8 describes a further characteristic of the observed finding (e.g., a location of the perforation). Accordingly, the inventive diagnostic encoding method and grid structure employed therein, provides an effective and uniform mapping of observed diagnostic findings to universally recognized and accepted codes representing portions of the human body.

As should be appreciated, the sub-descriptions included within aliquot 2 vary depending on the organ specified within aliquot 1. As described above, for example, aliquot 2 included an ear drum corresponding to the ear organ. Although not discussed herein, aliquot 2 corresponding to the ear organ also includes, as sub-descriptions, an exterior auditory canal, ossicles (three ear bones), labyrinth (balance canal), balance nerve, facial nerve, middle ear space, and Eustachian tube. As noted above, it is within the scope of the present invention to employ a plurality of aliquots (e.g., aliquots 3, 4, 5, etc.) to define component, sub-component, and sub-sub-component parts of each organ of the body. Similarly, it is within the scope of the present invention to employ a plurality of aliquots to define characteristics of symptoms and/or diagnostic findings.

It should also be appreciated that in accordance with one embodiment of the present invention a symbol is employed for indicating that an aliquot (e.g., representing an organ, component, a symptom, characteristic of a symptom, or a diagnostic finding) has been considered and found to “not apply” or “not applicable” or similar meaning. In this manner, for example, the inventive encoding method may indicate areas investigated and found not pertinent to a particular patient's case.

Additionally, it should be appreciated that the alphanumeric characters and other symbols used herein are for illustrative purposes only, as such, the present invention is not limited in this regard as any symbols may be substituted without departing from the broader aspects of the present invention. Furthermore, the present invention can be embodied as a spreadsheet, database or the like and used in connection with a computer processing device such as, for example, a personal computer, workstation, laptop, tablet computer, personal digital assistant or like portable computing device. The present invention may also be embodied in a paper chart as part of a method for recording the history of a patient's symptoms. It should also be appreciated that the inventive encoding method may be employed to store records and other information within the diagnostic environment. For example, filing cabinets, drawers, partitions, sub-partitions, and the like, may employ the encoding method described herein to classify information in a uniform manner to facilitate, for example, rapid retrieval of like information.

One or more embodiments of the present invention have been described. Nevertheless, it will be understood that various modifications may be made without departing from the spirit and scope of the invention. For example, many construction techniques and materials may be utilized. Accordingly, other embodiments are within the scope of the following claims.

What is claimed is:

1. A method for diagnosing and providing diagnostic and examination information, comprising:
storing a database on a computing device, the database including a first plurality of identifiers directed to anatomical structures and a second plurality of identifiers directed to diagnostic criteria by which caregivers evaluate patients;
displaying at least a portion of said first and second plurality of indicators on said computing device, each of said first and second indicators being selectable by a caregiver when diagnosing a patient;
storing said selected indicators in said database;
generating a code, via the cooperation of said database and said computing device, said code being comprised of said selected indicators; and wherein said code is indicative of an examination and diagnosis of said patient so that inputting said code into a computing device having said database stored thereon can cause an explanation of said examination and diagnosis to be displayed.

2. The method of claim 1, wherein the first plurality of identifiers includes:
a first subset of identifiers identifying each organ in the human body; and
a second subset of identifiers identifying component parts of each of the organs in the first subset.

3. The method of claim 2, wherein the first subset of identifiers is comprised of numeric characters, and wherein numeric characters representing organs on a first side of the body are comprised of even numeric characters and numeric characters representing organs on a second side of the body are comprised of odd numeric characters.

4. The method of claim 2, wherein the second plurality of identifiers includes:
a third subset of identifiers identifying symptoms relating to at least one of the first subset and second subset of identifiers.

5. The method of claim 4, wherein the second plurality of identifiers includes:
a fourth subset of identifiers identifying a first characteristics of the symptoms of the third subset.

6. The method of claim 5, wherein the fourth subset of identifiers describes a duration of the symptom as reported by the patient to the caregiver.

7. The method of claim 4, wherein the second plurality of identifiers includes:
a fifth subset of identifiers identifying a second characteristics of the symptoms of the third subset.

8. The method of claim 7, wherein the fifth subset of identifiers describes a characteristic of the start of the symptom as reported by the patient to the caregiver.

9. The method of claim 2, wherein the second plurality of identifiers includes:
a sixth subset of identifiers identifying an observed diagnostic finding relating to at least one of the first subset and second subset of identifiers.

10. The method of claim 9, wherein the second plurality of identifiers includes:
a seventh subset of identifiers identifying a first qualification of the observed diagnostic finding of the sixth subset.

11. The method of claim 10, wherein the seventh subset of identifiers describes a characteristic of a severity of the diagnostic finding observed by the caregiver.

12. The method of claim 9, wherein the second plurality of identifiers includes:
a eighth subset of identifiers identifying a second qualification of the observed diagnostic finding of the sixth subset.

13. The method of claim 12, wherein the eighth subset of identifiers describes a characteristic of a location of the diagnostic finding observed by the caregiver.

14. The method of claim 1, wherein the first plurality of identifiers and the second plurality of identifiers are comprised of alphanumeric characters and symbols.
